(19) United States
(12) Patent Application Publication
(10) Pub. No.: US 2005/0147524 A1
(43) Pub. Date: Jul. 7, 2005

(54) Sterile tubing termination assembly

(76) Inventor: Gerald G. Bousquet, Tyngsboro, MA (US)

Correspondence Address: CESARI AND MCKENNA, LLP, 88 BLACK FALCON AVENUE, BOSTON, MA, 02210, US

(21) Appl. No.: 10/752,475

(22) Filed: Jan. 6, 2004

(51) Int. Cl. A01N 1/00

(52) U.S. Cl. 422/28; 604/538

(57) ABSTRACT

A catheter termination assembly includes a catheter connection of the type having a coupling stem at one end, a catheter connection nipple at the opposite end, an axial lumen extending between those ends, and a transverse enlargement between the stem and the nipple. The assembly also includes cup-like end cap, the end cap having an end wall, and a skirt extending from the end wall. The skirt is sized to engage around the enlargement and interfitting surfaces on the enlargement and interior wall of the skirt enable the skirt to be releasably coupled to the enlargement so as to define a fluid-tight chamber encircling the stem. A wall of the end cap has an opening filled by a needle-penetrable, self-sealing septum by which a sterilizing agent may be injected into the chamber so as to immerse the stem in the sterilizing agent.

### BACKGROUND OF THE INVENTION

This invention relates to apparatus for inhibiting or eliminating the colonization by microorganisms of in-dwelling medical devices and the subsequent occurrence of infections associated with same.

1. Field of the Invention

Infections associated with medical care are a major cause of morbidity and mortality. These infections are typically very costly to manage and may be associated with a variety of adverse outcomes including death. Common types of infections that occur in an acute care setting include pneumonia, wound infection and blood stream infection. Often these infections develop from the use of invasive devices in patients with limited resistance to infection as a result of the underlying illness or drug therapy. Also, because many strains of microorganisms acquired in the hospital are resistant to commonly used antibiotics, it is often very difficult and costly to treat these infections.

Medical catheter or device-related infections such as those associated with intravenous, intraarterael, dialysis and other types of medical catheters or implanted medical devices usually result because of a breach of natural protective mechanisms present at sites such as the skin. In these situations, the development of infection is believed to involve the following steps:
1. Microorganisms colonize and multiply in a patient's skin. There is a microbial flora normally present on the skin. When a patient is admitted to a healthcare setting, organisms from this environment (typically resistant to antimicrobials) become part of the patient's flora soon after admission;
2. After insertion of the catheter or other medical device, these organisms continue to multiply and begin to colonize the surface of the catheter or device;
3. As the organisms continue to multiply, they spread on the external and/or internal surfaces of the catheter or device and eventually enter normally sterile tissues (e.g. blood vessels, abdomen, etc.). This process is facilitated by the development of a biofilm (which consists of proteins from the blood or tissue fluid and other debris) on the surface of the devices, and
4. Once in these normally sterile sites, the organisms continue to multiply and the development of a clinical infection may result.

It has been known for many years that staphylococcus aureus, for example, nearly always can be found to colonize the patients that develop infection with this organism, colonization meaning the presence of the organism without evidence of infection.

One of the most important sites for colonization is the anterior part of the nose (anterior nares). Individuals that develop a staphylococcal infection at the site of an in-dwelling catheter or device usually have nasal colonization with this organism.

2. Description of the Prior Art

One device category that is particularly susceptible to biological contamination are the fittings or connectors at the ends of catheters implanted in patients to provide prolonged or repeated access to the internal organs of those patients. For example, a central venous catheter (CVC) is used to access a patient's venous system for the administration of intravenous (IV) fluids, antibiotics and chemotherapy. Catheters are also implanted in patients who require repeated access to the peritoneum for peritoneal dialysis. Such a catheter exits the patient's body at the skin line usually through a standard transcutaneous access device. A short length of catheter resides outside the body and is terminated by an end fitting, often a male Luer connector or the like. When treating the patient, that connector is coupled to a mating female connector at the end of a length of tubing leading to a dialysis machine, fluid administration set or the like. When the therapy session is completed, the mated connectors are decoupled and the connector at the end of the catheter extending from the patient is closed by an end cap which is basically a blind or dead end version of the female connector. Invariably prior to affixing the end cap, the male connector is sterilized by wiping it down with a disinfectant such as alcohol, bleach or betadyne. However, I have found through testing that even after such a wipedown, appreciable biological contamination still exists on the connector due to the fact that the connector has an exterior thread or crannies and crevasses which are difficult to reach with a disinfectant wipe.

Resultantly, when the connector is again coupled to the mating connector of the dialysis machine or administration set, biological contaminants may be entrained in the fluid flow to the patient giving rise to infection and possible sepsis.

A variety of techniques have been used in an attempt to reduce the frequency of the infections described above. These have included impregnating the catheter connectors with antibiotic or photodynamic substances, incorporating silver or silver compounds in the connectors and irradiating the devices with infrared or ultraviolet light. Each of these prior techniques has, to some extent, reduced the frequency of infection. However, none of them is ideal. An ongoing concern is that microorganisms have the potential to develop resistance to antimicrobials incorporated into the catheter or device in an attempt to prevent infection. Thus, there is an on-going need for an improved mechanism for sterilizing the end fittings of in-dwelling medical catheters.

### SUMMARY OF THE INVENTION

Accordingly, it is an object of the present invention to provide a sterile termination assembly for medical tubing.

Another object of the invention is to provide a sterile catheter connector assembly.

Still another object is to provide an assembly such as this which is relatively easy and inexpensive to make in quantity.

Yet another object of the invention is to provide a device for maintaining the sterility of the connector of an in-dwelling medical catheter.

Other objects will, in part, be obvious and will, in part, appear hereinafter.

The invention accordingly comprises the features of construction, combination of elements and arrangement of parts which will be exemplified in the construction hereinafter set forth, and the scope of the invention will be indicated in the claims.

Briefly, my sterile catheter or tubing termination assembly comprises a connector which, in use, is secured to the exposed end of a catheter or length of tubing implanted in a patient usually by way of a transcutaneous access device; see e.g. my U.S. Pat. No. 5,662,616. The connector may have a more or less standard configuration, i.e. like that of a Luer connector or screw connector commonly used in the medical industry. Often the connector that is attached to the catheter implanted in the patient is a male connector which is adapted to be coupled to a corresponding female connector having the same configuration. The present connector is more or less standard in that it has a tubular body with a coupling stem at one end, a catheter nipple at the other end and a lumen extending between said ends. The coupling stem is the part of the connector most prone to transferring biological contamination to the patient.

The connector of this invention differs from a standard medical connector only in that its body has an enlargement or flange between its ends and the periphery of that enlargement or flange is threaded or otherwise shaped to make a connection with an end cap to be described now.

The other component of the termination assembly is a cup-like end cap which may be retrieved from a sterile package. The end cap has an internally threaded wall which screws onto or otherwise interfits with the connector enlargement or flange so that the end cap and connector from a fluid tight enclosure or chamber containing the coupling stem of the connector.

In accordance with the invention, a small opening is provided in a wall of the end cap and that opening is closed by a needle-penetrable septum so that a sterilizing agent can be injected into the chamber thereby immersing the connector stem in that agent for a long enough time to completely sterilize or disinfect that stem. Preferably, a small check value is provided in a wall of the end cap to vent the chamber as that is being filled with the sterilizing agent.

In one embodiment of the invention, the sterilizing agent is a chemical disinfectant such as alcohol, bleach, betadyne or the like. Therefore, in order to prevent the sterilizing agent from entering the lumen of the connector body, the end wall of the end cap is formed with an integral post which extends into the end of the connector stem and blocks the opening into that lumen when the cap is coupled to the connector.

In a second invention embodiment, that post is not required because the chamber surrounding the connector stem is filled with a biocompatible sterilizing agent such as saline solution. The volume of solution in that chamber is then heated by microwave energy or by other means to a temperature sufficient to kill any microbials resident on the connector stem.

In both embodiments, the end cap and its now sterile contents may remain on the connector until it is necessary to couple that connector to a mating connector at the end of a catheter leading to a dialysis machine, administration set or other therapeutic device. When that time arrives, the termination is pointed downward and the end cap is released from the connector with the sterilizing agent remaining in the end cap. The end cap may be thrown away or sterilized for reuse. The sterile connector stem may then be coupled to its mate in the usual way with assurance that there has been no bacterial formation on the connector stem during its period of nonuse.

### BRIEF DESCRIPTION OF THE DRAWINGS

For a fuller understanding of the nature and objects of the invention, reference should be made to the following detailed description, taken in connection with the accompanying drawings, in which:

FIG. 1 is a diagrammatic view showing a pair of in-dwelling catheters which are terminated according to the invention;

FIG. 2A is a side elevational view with parts in section, on a much larger scale, showing the components of FIG. 1 the termination in greater detail;

FIG. 2B is a sectional view taken along line 2B-2B of FIG. 2A, and

FIG. 3 is a view similar to FIG. 2A showing a second termination embodiment.

### DETAILED DESCRIPTION OF ILLUSTRATIVE EMBODIMENTS

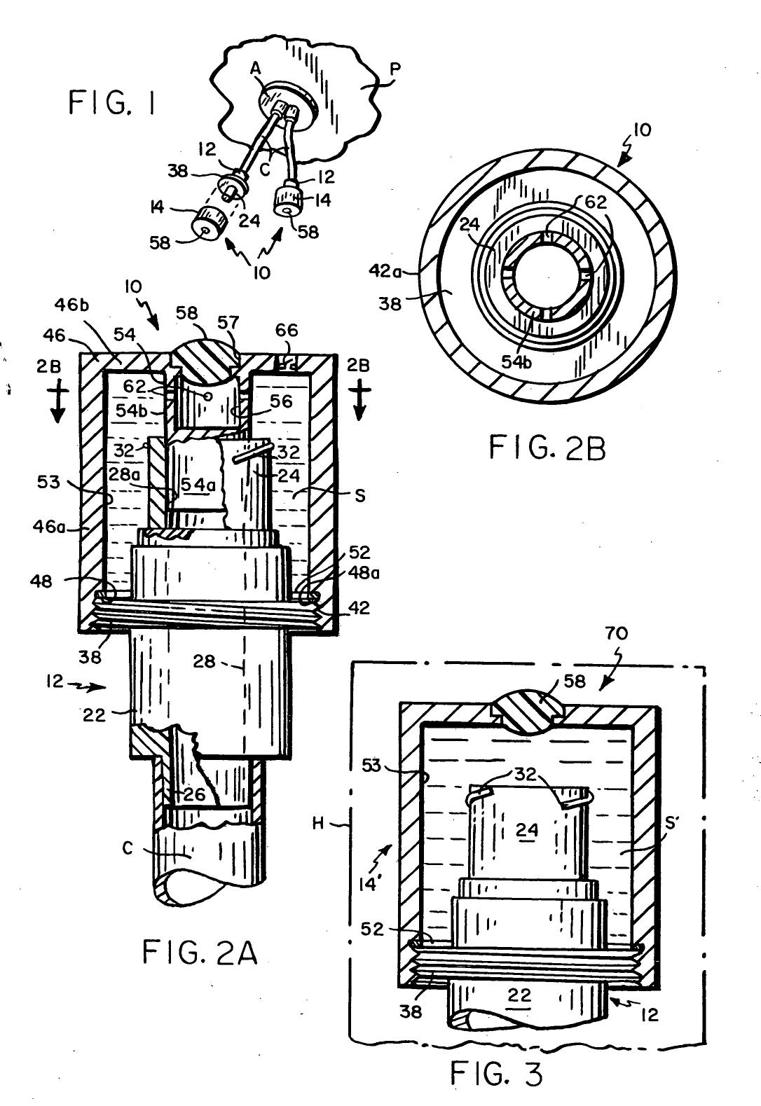

Refer now to FIG. 1 which illustrates a skin area of a patient P where catheters C are implanted in the patient by way of a transcutaneous access device A such as the one shown in the above-identified patent. Each catheter C is terminated by a termination assembly shown generally at 10. Each assembly includes a connector 12 and an end cap 14 which may be releasably secured to the connector, the end caps being shown in both unassembled and assembled positions.

In a typical application, the catheters C may be connected to a dialysis machine in order to treat a patient with chronic kidney failure. In that event, with the end caps 14 removed from the respective connectors 12, those connectors are coupled to the mating connectors (not shown) at the ends of tubing leading to and from the dialysis machine. When the connectors 12 are decoupled from their mates, they may be protectively enclosed by end caps 14 as will be described.

Referring now to FIGS. 2A and 2B, each connector 12 may be a more or less standard connector of the type used to terminate catheters in the medical industry, e.g. a screw-type connector as shown or a Luer connector. The illustrated connector 12 attached to each catheter C happens to be a male screw-type connector and includes a tubular main body 22 having a cylindrical coupling stem 24 extending from one end and a nipple 26 extending from the opposite end. An axial lumen 28 extends the entire length of the connector. An end segment of catheter C is engaged over nipple 26 and sealed thereto so that the catheter lumen is in fluid communication with connector lumen 28. The coupling stem 24 has exterior threads 32 of the type commonly found on screw-type connectors of this general type.

The segment 28a of the lumen 28 extending through the stem 24 is slightly tapered as is customary for connectors for this type and is adapted to be coupled to an internally threaded female connector (not shown). Since that female connector is conventional and does not form part of the invention, it will not be described in detail. Suffice it to say that the female connector includes a tube with a cup-like skirt surrounding the tube. A segment of the tube within the skirt is exteriorly tapered and the rim of the skirt is internally threaded so that when the skirt is screwed onto stem 24, the tube is wedged into the lumen segment 28a of stem 24 so that a seal is made between the tube and the stem. The opposite end of that tube is connected to one end of a tube leading to or from an external device, e.g. a dialysis machine.

The only material difference between connector 12 and a standard connector of the same type is that it has a circular enlargement or flange 38 between its ends which is larger (in radius) than the flange or enlargement found on the conventional connectors. Furthermore, that enlargement or flange 38 has surfaces at its periphery that can interfit with opposing surfaces on end cap 14. In the illustrated assembly 10, those interfitting surfaces are screw threads 42.

Still referring to FIGS. 2A and 2B, end cap 14 is a cup-like member 46 having a cylindrical side wall or skirt 46a and a discoid end wall 46b. The side wall 46a of cap 14 has an internally threaded counterbore 48 so that the end cap can be screwed onto the threaded flange 38 of the connector 12. The shoulder 48a at the bottom of counterbore 48 may be covered by a soft plastic film 52 to provide a seal when the end cap 14 is screwed onto the connector 12. Thus, when end cap 14 is secured to connector 12, a fluid-tight chamber 53 is formed which completely surrounds connector stem 24.

Of course, other means for releasably connecting cap 14 to connector 12 may be envisioned. For example, flange 38 may be formed with a groove and the side wall of counterbore 48 may have an interior rib which snaps into that groove when cap 14 is seated on flange 38, or vice versa. Of course with this type of non-screw connection, the enlargement 38 and skirt 46a do not have to be circular.

As best seen in FIG. 2A, an axial post 54 extends down from the end wall 46b of end cap 14 at the center of that wall. Post 54 has a slight external taper so that when the end cap 14 is screwed onto connector 12, a segment 54a of the post projects into stem 24 and wedges against the wall of lumen segment 28a providing a fluid-tight seal between the post and stem 24.

While the end segment 54a of post 54 that seats in the lumen segment 28a is solid, the post segment 54b outside the stem 24 is hollow to define a small antechamber 56 between the post segment 54a and the end cap end wall 46b. Also, a small opening 57 is formed in end wall 46b in line with antechamber 56 and that opening is closed by a needle-penetrable septum 58. Small holes 62 are provided in the side wall of the tubular post segment 54b establishing fluid communication between antechamber 56 and the larger chamber 53 containing connector stem 24.

Thus, using a hypodermic needle (not shown) inserted through septum 58, a liquid sterilizing agent S can be injected into antechamber 56. That liquid will flow through the holes 62 into chamber 53 filling that chamber. Preferably, in order to allow the escape of air from chamber 53 during the filling process, a small check valve 66 may be provided in a wall of end cap 14, e.g., end wall 46b.

During the filing process, the antechamber collects any debris due to penetration of the septum and the bottom wall of the antechamber functions as a needle stop.

In some applications, the antechamber may not be required. In that event, the post 54 may be solid from end to end and the septum 58 located elsewhere on the end cap so that it is not in line with that post.

In order to use the termination assembly 10, after a particular therapy session has ended, connector 12 may be decoupled from its mating connector leading to the external therapy device, e.g., a dialysis machine. An end cap 14 may then be removed from its sterile package and screwed onto the flange 38 of the connector 12 until a seal is made at the boundary of the end cap and the flange, i.e., at film 52. Then, a sterilizing agent S such as alcohol, bleach or betadyne may be injected through septum 58 into antechamber 56. The injected sterilant will flow through holes 62 and fill the chamber 53 thereby completely immersing the working end of the connector 12, including its stem 24, in the sterilant. That portion of the connector may remain immersed in sterilant until the next therapy session. At that time, with the end cap pointed downward, the end cap may be unscrewed from connector 12 so that the sterilizing agent does not spill out of the end cap. The connector 12 may then be immediately coupled to a pre-sterilized mating connector at the end of the catheter leading to the external therapy device. The end cap 14 and its contents may be thrown away or the end cap may be emptied and sent to a sterilization facility so that it can be reused.

Refer now to FIG. 3 which illustrates another termination assembly embodiment, shown generally at 70, which utilizes a biocompatible sterilizing agent S′ such as saline solution or distilled water. Since most of its parts are similar to those of termination 10, those similar parts of termination 70 carry the same identifying numerals.

In this case, the termination includes an end cap 14′ which lacks post 54 and perhaps also check valve 66. This is because the chamber 53 within the end cap 14′ is filled with a biocompatible sterilizing agent S which may pass into catheter C without hurting the patient P. The volume of sterilant S′ inside chamber 53 is then heated by ternal heating means H to a temperature sufficient to kill any bacteria or microbials on the portion of connector 12 inside that chamber. The external heater H may be a small microwave or RF heater or an electric heater. In any event, the heater heats the volume of liquid inside chamber 53 to a high enough temperature quickly enough to kill the microbials on the connector stem without appreciably heating any saline that may find its way into catheter C. Once sterilized, the contents of the end cap 14′ remain sterilized at least until the end cap is removed from the connector 12.

The components of termination assemblies 10 and 70 are molded of a suitable medical grade plastic material able to withstand sterilization and are relative inexpensive to make in quantity. Therefore, the overall cost of the termination assembly is comparable to that of a conventional male/female connector assembly.

It will thus be seen that the objects set forth above among those made apparent from the preceding description are efficiently attained.

Also, since certain changes may be made in the above constructions without departing from the scope of the invention, it is intended that all matter contained in the above description or shown in the accompanying drawings shall be interpreted as illustrative and not in a limiting sense.

It is also to be understood that the following claims are intended to cover all of the generic and specific features of the invention described herein.

1. A catheter termination assembly comprising
a catheter connection of the type having a coupling stem at one end, a catheter connection nipple at the opposite end and an axial lumen extending between said ends, said connection also including a circular enlargement between said stem and said nipple, said enlargement having a threaded circumferential surface, and
a cup-like end cap including an end wall and a cylindrical skirt extending from the end wall, said skirt having an interiorly threaded end segment sized to threadedly engage around said enlargement so that the end cap may be screwed to the enlargement to form therewith a fluid-tight chamber encircling said stem, an opening in the end wall, and a needle-penetrable self-sealing septum closing said opening by which a sterilizing agent may be injected into said chamber so that said stem is immersed in the sterilizing agent.

2. The assembly defined in claim 1 and further including a sterilizing agent in said chamber.

3. The assembly defined in claim 1 and further including a valve mounted in a wall of said end cap for venting said chamber.

4. The assembly defined in claim 1 wherein the threaded end segment of the skirt has a larger inside diameter then the remainder of the skirt and defines a flat seating surface for the end cap.

5. The assembly defined in claim 1 and further including an axial post extending from the end wall and sized to project into said stem and block said lumen when the end cap is screwed onto said enlargement.

6. The assembly defined in claim 5 wherein said opening and said septum are radially displaced on the end wall from said post.

7. The assembly defined in claim 5 wherein
the post has a solid segment which projects into the stem, and
a tubular segment outside the stem which defines an antechamber in fluid communication with both said opening and said chamber.

8. The assembly defined in claim 5 and further including a chemical sterilizing agent filling said chamber.

9. The assembly defined in claim 8 wherein the sterilizing agent is selected from the group consisting of alcohol, bleach and betadyne.

10. The assembly defined in claim 2 wherein the sterilizing agent is a biocompatible liquid.

11. The assembly defined in claim 10 and further including a heater sized to receive the screwed-together connector and end cap for heating the contents thereof to a temperature sufficient to kill organic contaminants on said stem.

12. The assembly defined in claim 1 wherein said connector and said end cap are of a medical grade plastic material able to withstand sterilization.

13. A catheter termination assembly comprising
a catheter connection of the type having a coupling stem at one end, a catheter connection nipple at the opposite end and an axial lumen extending between said ends, said connector also including a transverse enlargement between said stem and said nipple, and
a cup-like end cap, said end cap including an end wall and a skirt extending from the end wall, said skirt being sized to engage snugly around said enlargement, said enlargement and said skirt having interfitting surfaces enabling the skirt to be releasably coupled to the enlargement so as to define a fluid-tight chamber surrounding said stem, an opening in a wall of the end cap, and a needle-penetrable self-sealing septum closing said opening by which a sterilizing agent may be injected into said chamber so as to immerse the stem in the sterilizing agent.

14. The assembly defined in claim 13 and further including a sterilizing agent at least partially filling the chamber.

15. The assembly defined in claim 14 wherein the sterilizing agent comprises a biocompatible liquid.

16. The assembly defined in claim 15 wherein the sterilizing agent is saline solution or distilled water.

17. The assembly defined in claim 15 and further including a heater for heating the contents of said chamber to a temperature sufficient to kill microbials on said stem.

18. The assembly defined in claim 14 wherein the sterilizing agent is a chemical disinfectant, preferably alcohol, bleach, or betadyne.

19. The assembly defined in claim 18 and further including an axial post extending from the end wall and sized to project into said stem and block said lumen when the skirt is coupled to said enlargement.

20. The assembly defined in claim 19 wherein said opening and said septum are laterally displaced on the end wall from said post.

21. The assembly defined in claim 19 wherein
the post has a solid segment which projects into the stem, and
a tubular segment outside the stem which defines an antechamber in fluid communication with both said opening and said chamber.
